(19) United States
(12) Patent Application Publication
(10) Pub. No.: US 2020/0367731 A1
(43) Pub. Date: Nov. 26, 2020

(54) MULTI CAMERA MEDICAL SURGERY ILLUMINATING DEVICE WITH A CHANGING DIAMETER

(71) Applicant: 270 SURGICAL LTD., Netanya (IL)

(72) Inventors: Avraham LEVY, Kfar Shmaryahu (IL); Moshe LEVI, Ganey Tikva (IL); Golan SALMAN, Atlit (IL)

(21) Appl. No.: 16/638,156

(22) PCT Filed: Aug. 7, 2018
(86) PCT No.: PCT/IL2018/050873

§ 371 Date: Feb. 11, 2020

Related U.S. Application Data
(60) Provisional application No. 62/546,581, filed on Aug. 17, 2017.

(51) Int. Cl. A61B 1/00; A61B 1/05; A61B 1/313

(52) U.S. Cl. CPC A61B 1/00181; A61B 1/3132; A61B 1/051

(57) ABSTRACT

The subject matter discloses a medical imaging device, comprising a rigid elongated member having a first cross-sectional diameter, a distal tip having a second cross-sectional diameter, wherein the second cross-sectional diameter is bigger than the first cross-sectional diameter by at least 0.02 millimeter, said distal tip comprises a front camera located on a front planar surface of the distal tip and a first side camera located on a first lateral surface of the distal tip.

### FIELD OF THE INVENTION

The present invention generally relates to the field of medical instruments designed to capture images from inside the patient's body.

### BACKGROUND OF THE INVENTION

A laparoscope is a device utilized to perform operations in the abdomen or pelvis through small incisions with the aid of a camera. It can either be used to inspect and diagnose a condition or to perform surgery. In some cases, procedures which involve inspection of a region inside confined area or a specific body cavity or organ, may also involve an endoscope.

A laparoscope is likely to be assembled in an elongated tubular member in which the camera is located, as well as all the electrical circuitry. In general, the procedure of laparoscopy starts with a small incision made near the belly and the abdomen is filled with CO2 gas. The CO2 lifts the abdomen away from the internal organs. Then a laparoscope is inserted into the abdomen and provides the surgeon with the needed view of the internal organs. In some cases, a trocar is inserted into the abdomen through the incision and the laparoscope is pushed into the abdomen through a cannula of the trocar.

The laparoscope may have an elongated member which enables maneuvering the laparoscope in the patient's body. The elongated member may be rigid, for protecting the circuitry and sensors. In medical procedures, the used laparoscope may comprise more than one camera located at the front of the laparoscope. Thus, the cameras, the optical components and the electrical circuitry may be required to be placed in a relatively wide laparoscope. In such cases, the challenge is to maintain the narrowest incision possible. In some cases, a separate trocar is used, the cannula of the trocar may limit the width of the multi-camera laparoscope.

### SUMMARY OF THE INVENTION

The present invention discloses a thin medical imaging device comprising two section members directly connected. Said two section members may be an elongated rigid shaft and a distal tip. The distal tip comprises the optical gear required for the medical procedures, and is connected directly to the rigid shaft. The optical gear located in the distal tip can comprise cameras, lenses and light sources required for the camera functioning. The distal tip can be provided in a changing diameter, wherein the maximum diameter is approximately 10.0 to 20 millimeters, and the minimal diameter is approximately 2.5 to 15.0 millimeters. In some cases, the rigid shaft may be provided with a narrower diameter than the distal tip, such that the rigid shaft may be connected to the narrowest part of the distal tip. In some cases, the diameter of the distal tip gradually increases from the shaft diameter to the maximal diameter of the distal tip. In some embodiments of the disclosed subject matter, the distal tip may comprise an inclined surface beginning at the part of the distal tip with the broadest diameter and inclines down to the narrowest part of the distal tip. In some cases, the inclined surface may be replaced with a graded edge allowing to connect the distal tip to the rigid shaft which comprises a narrower diameter than the distal tip.

The distal tip also comprises a front camera located on a front planar surface of the distal tip and a second side camera located on a first lateral surface of the distal tip. In some cases, the medical imaging device may also comprise a first side camera located on a second lateral surface of the distal tip.

In possible embodiments of the disclosed subject matter, the distance between the lens center of the second side camera and the lens center of the front camera may be shorter than the distance between the lens center of first side camera and the lens center of the front camera. In other possible embodiments of the disclosed subject matter, the distance between the lens center of the second side camera and the lens center of the front camera may be longer than the distance between the lens center of first side camera and the lens center of the front camera. In some cases, the working distance of the front camera, the second side camera and the first side camera may be in a range of 1-150 millimeters. I some cases, the second side camera and the first side camera have a horizontal field of view of between 60-160 degrees.

In some cases, the distal tip may further comprises at least one aperture shaped for securing the first side camera, the front camera and the second side camera. In some cases, each aperture is associated with a single camera. That is, one aperture for the first side camera, one aperture for the front camera and one aperture for the second side camera. In some cases, said apertures may have opaque walls with transparent apertures located near the front camera, the first side camera, and the second side camera. The distal tip may further comprise a front illumination module for illuminating the area captured by the front camera a second side illumination module for illuminating the area captured by the second side camera, and a first side illumination module for illuminating the area captured by the first side camera. Thus, the front illumination module may comprise two illumination modules on both sides of the front camera, wherein one illumination module may be bigger than the other in cases, wherein the front camera may not be positioned in the center of the front surface.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 demonstrates a medical imaging device comprising at least one camera and a changing diameter, according to exemplary embodiments of the disclosed subject matter;

FIG. 2 demonstrates an upper cross section of a medical imaging device provided in a changing diameter and comprises three cameras, according to exemplary embodiments of the disclosed subject matter;

FIG. 3 shows an upper cross section of a medical imaging device with a changing diameter comprises one front camera and two side cameras, according to FIG. 2;

FIG. 4 shows a schematic view of a medical imaging device with a changing diameter comprises one front camera and two side cameras, according to exemplary embodiments of the disclosed subject matter,

FIG. 5 shows a schematic view of a medical imaging device with a distal tip comprising a graded edge, according to exemplary embodiments of the disclosed subject matter;

FIG. 6 demonstrates the field of view of three cameras in a medical imaging device with a changing diameter and having a symmetry of overlap between the FOVs, according to exemplary embodiments of the disclosed subject matter, end;

FIG. 7 demonstrates a medical imaging device comprising a one-piece medical imaging device with a changing diameter comprising the optical gear required to the operation of the medical imaging device, according to exemplary embodiments of the disclosed subject matter.

### DETAILED DESCRIPTION OF THE INVENTION

The present invention discloses a thin medical imaging device which can be utilized in cases the medical imaging device is required to pass through an incision in the body and perform medical procedures at the internal organs. Such a medical imaging device comprises two or more cameras designed to aid medical procedures such as inspection or surgery procedures in the abdomen or pelvis through small incisions. The medical imaging device is provided with at least two diameters wherein some sections of the medical imaging device may have a wide diameter, such as the distal tip, allowing a required space for the optical gear, and some other sections of the medical imaging device may be with a narrow diameter allowing the medical imaging device to be threaded through relatively small incision at the human body, for example being mounted at least partially in a trocar, having a predefined internal diameter. Yet, the rigid shaft may be placed in the trocar during the medical procedure, while the distal tip is in the patient's body, capturing images. The distal tip of the imaging device comprises the optical gear only has to pass through the trocar, which has some elasticity for penetrating the wider tip. The wider tip provides more space for the optical gear, enabling better sensors, lenses and thus better data captured by the imaging device. Inserting the device via a trocar enables using a relatively small incision at the human body and facilitate the medical procedure in case a gas is required to be filled in a cavity at the human body. The small incision may ease the creation of a sealant to prevent a gas leak from the incision. The term diameter refers to the cross sectional diameter of the rigid shaft and the cross sectional diameter of the distal tip.

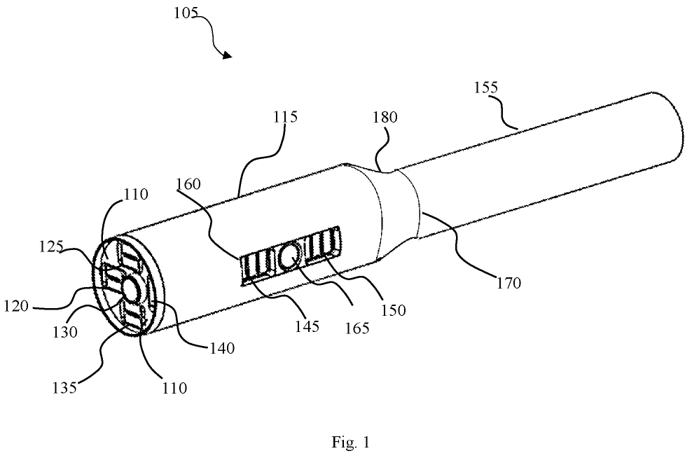

FIG. 1 demonstrates a medical imaging device comprising at least one camera and a changing diameter, according to exemplary embodiments of the disclosed subject matter. FIG. 1 shows a medical imaging device 105 comprising a rigid shaft 155 designed to be directly connected to a distal tip 115. The distal tip 115 may comprise an inclined surface 180 allowing to connect the distal tip 115 to the rigid shaft 155 provided in a narrower diameter than the diameter of the distal tip 115.

The distal tip 115 may also comprise a seamline 170 which outlines the connection line between the rigid shaft 155 and the inclined surface 180 of the distal tip 115. In some cases, the rigid shaft 155 and the inclined surface 180 may be connected by an adhesive material which seals the connection at the seamline 170. In some other cases, the rigid shaft 155 and the inclined surface 180 may be connected by soldering. In possible embodiments of the disclosed subject matter, the rigid shaft 155 and the inclined surface 180 may be connected by a screwing mechanism which fastens the rigid shaft 155 and the distal tip 115 together.

The distal tip 115 may function as a multi-camera section member designed to house at least one camera. In some cases, the cameras may be positioned at the front end of the distal tip 115, defined as planar surface 110. Additional cameras may be located at the lateral round surface of the distal tip 115. The distal tip 115 may also comprise an aperture 160 shaped to house the second side camera 165 and provide the opening required for the field of view of the second side camera 165. In some cases, the aperture 160 may be covered by a transparent layer, such as glass or plastic, to isolate the second side camera 165 from the patient's tissue. In some other cases, aperture 160 may be covered by an optical window or more than one optical window.

In some embodiments of the disclosed subject matter, the distal tip 115 may comprise a first side camera (not shown). The first side camera may be located at the opposite side of the distal tip 115. The aperture 160 also enables emission of light from side illuminator modules 150, and 145 which provide the light source of the second side camera 165. In some cases, the light may be emitted by dedicated illuminators such as light-emitting diode, also known as LED.

The distal tip 115 may also comprise a front camera 130 situated on a planar surface 110 which can house the front camera 130 and provide the opening required for the field of view of the front camera 130. The planar surface 110 also comprise front illuminators 120, 125, 135, and 140 which provide the required source of light for front camera 130. In another embodiment, the number and location of front illuminators may vary. For example, less than 4 illumination modules or more wherein each illumination module has 1 or more LEDs and may emit different light spectrums.

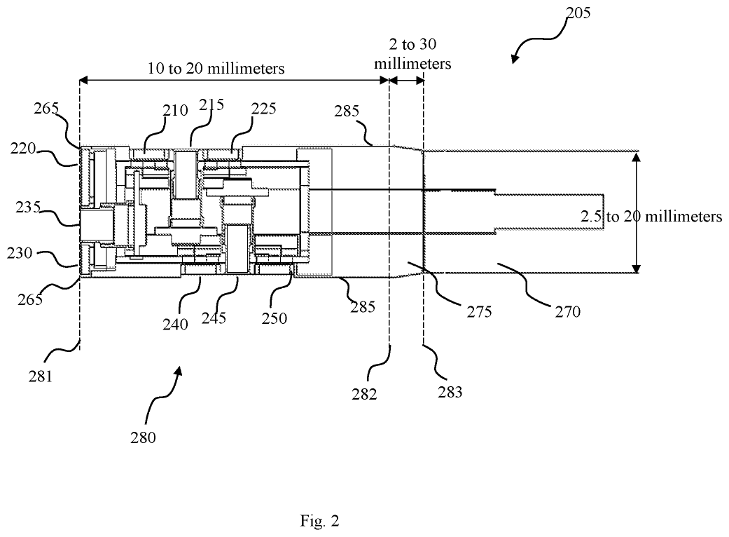

FIG. 2 demonstrates an upper cross section of a medical imaging device provided in a changing diameter and comprises three cameras, according to exemplary embodiments of the disclosed subject matter. FIG. 2 shows a medical imaging device 205 comprising a distal tip 280. The distal tip 280 comprises three cameras, front camera 235, second side camera 245, and first side camera 215. The medical imaging device 205 also comprises a tip section 265 housing the front camera 235, wherein the front camera 235 comprises a lens assembly for capturing a front field of view. In some exemplary cases, the field of view of the front camera 235 may be of at least 60 degrees, at least 80 degrees, at least 100 degrees and a working distance of approximately 1 to 30 millimeters, approximately 15 to 150 millimeters. The front camera 235 can be positioned on a surface of the tip section 265 of the distal tip 280.

The distal tip 280 further comprises a second side camera 245 which can be positioned within the medical imaging device 205 such that the center of said camera may be approximately 5 to 25 millimeters from the tip section 265 of the medical imaging device 205. The field of view of the second side camera 245 may be at least 60 degrees, at least 80, at least 100 degrees, with a working distance of approximately 1-30 millimeters, approximately 15 to 150 millimeters. The distal tip 280 may also comprise two second side illumination modules 240, and 250 which can be LEDs emitting light required for the operation of the second side camera 245. In some cases, the number of illumination modules may be more than two, wherein each illumination modules may include 1 or more LEDs and may emit different light spectrums.

The first side camera 215 may be positioned at the opposite side of the second side camera 245 such that the two cameras, second side camera 245 and first side camera 215, may point at directions essentially opposing to one another. The center of the first side camera 215 camera may be approximately 5 to 25 millimeters from the tip section 265 of the distal tip 280. The field of view of the first side camera 215 may be at least 60 degrees, at least 80, at least 100 degrees, with a working distance of approximately 1 to 30 millimeters, approximately 15 to 150 millimeters. The medical imaging device 205 may also comprise two first side illumination modules 225, and 210 which can be LEDs emitting light required for operation of the second side camera 215. In some cases, the number of illumination modules may be more than two, wherein each illumination module may include 1 or more LEDs and may emit different light spectrums.

The front camera 235 may be situated at the front surface of the distal tip 280. The front camera 235 may comprise a lens assembly providing a front field of view of at least 60 degrees, at least 80, at least 100 degrees and a working distance of approximately 1 to 30 millimeters, approximately 15 to 150 millimeters. The medical imaging device 205 may also comprise two or more front illumination modules. FIG. 2 shows illuminator modules 220 and 230 which can comprise LEDs emitting light required for operation of the front camera 235. In some embodiments of the disclosed subject matter, the light emitted by the LEDs may be a white light. In some other cases, a portion of the light sources of the medical imaging device 205 may be at different colors at the visible light spectrum. For example, the light source of the medical imaging device 205, may comprise LEDs emitting other colors such as blue, red, yellow, green, or any combination thereof. In some cases, the light emitted by the LEDs may be at the spectrum of the non-visible light. For example, a light source can provide a light at the infrared spectrum, ultra-violate, x-ray, and the like. The illumination modules 220, 235, 225, 240, 250 and 210 may receive electrical power via a cable placed in the rigid shaft 155.

In some embodiments of the disclosed subject matter, the distal tip 280 may be provided in the two sections with different diameters. Thus, one section of the distal tip 280 may be provided in a cylindrical shape with plain surface 285, and in a length between 10 to 20 millimeters. The cylinder-shaped section with the plain surface 285 is shown between an axis 281 and an axis 282. In one embodiment, axes 281, 282 and 283 are perpendicular to the longitudinal axis of the rigid shaft and the distal tip 280. A second section of the distal tip 280 is a section having an inclined surface 275. The length of the second section may be between 2 to 30 millimeters. The second section with inclined surface 275 is shown between the perpendicular axis 282 and the perpendicular axis 283. In such cases, the section with the inclined surface 275 may be connected directly to the rigid shaft 270 which may have a width in the range of 2.5 to 15 millimeters. Thus, in such cases, the maximum diameter of the distal tip may be approximately 10.0 to 20 millimeters, and the minimal diameter may be approximately 2.5 to 15.0 millimeters. In another embodiment, axis 281 may be titled to form an angle of less than 10 degrees from the longitudinal axis of the of the rigid shaft and the distal tip 280.

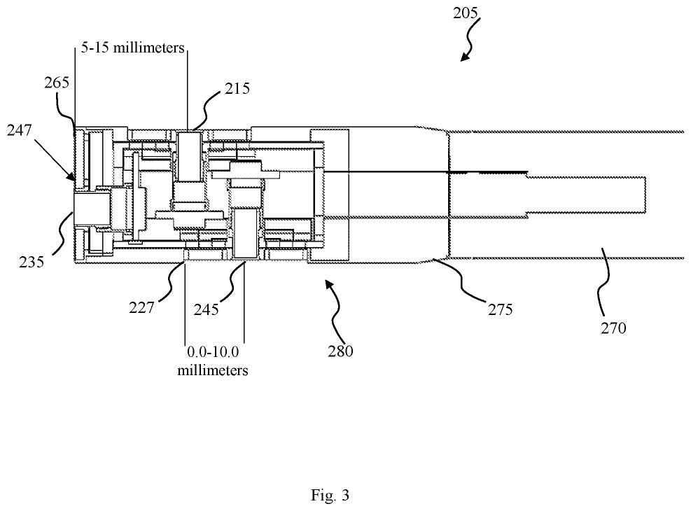

FIG. 3 shows an upper cross section of a medical imaging device with a changing diameter comprises one front camera and two side cameras, according to FIG. 2. FIG. 3 shows a medical imaging device 205 comprising three cameras located at the distal tip 280. The front camera 235 positioned at the front planar surface 247 located at tip section 265 of the distal tip 280. In some cases, the front camera 235 may be situated with a bias to one of the sides of the front planar surface 247. In another case, the front camera 235 may be situated in the center of front planar surface 247. The medical imaging device 205 also comprises a first side camera 215 located such that the center of first side camera 215 may be located approximately 0.0 to 10.0 millimeters from tip section 265. Second side camera 245 is located such that the center of second side camera 245 may be located approximately 0.0 to 10.0 millimeters from the center of first side camera 215 to the imaginary line continuing from the central of the first side camera 215 to point 222.

In some embodiments of the disclosed subject matter, the distal tip 280 may be provided in an inclined surface 275 at the section connected to the rigid shaft 270. In such cases, the distal tip 280 may be connected to the rigid shaft having in a narrower diameter relative to the distal tip 280.

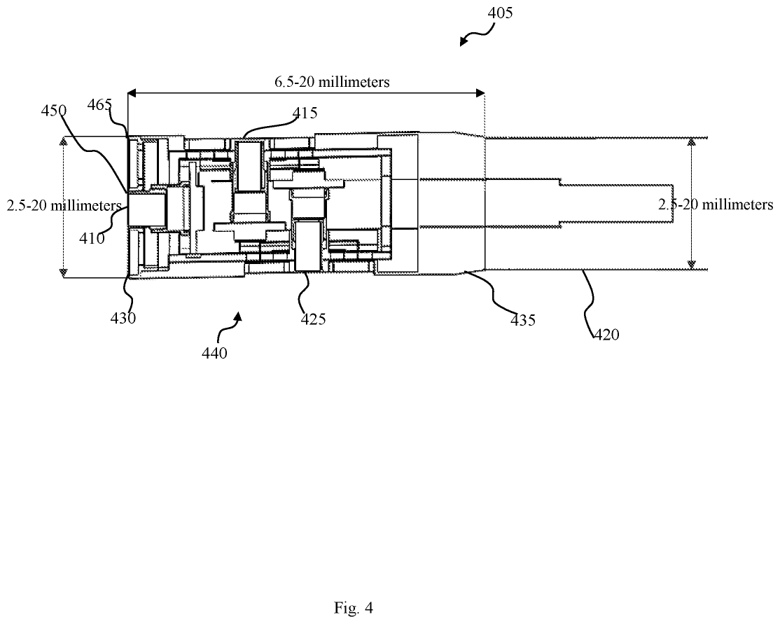

FIG. 4 shows a schematic view of a medical imaging device with a changing diameter comprises one front camera and two side cameras, according to exemplary embodiments of the disclosed subject matter. FIG. 4 shows a medical imaging device 405 comprising a distal tip 440 designed to house three cameras. The distal tip 440 may house a front camera 410 positioned essentially at the center of the front planar surface 450. The distal tip 440 further comprises a second side camera 425 located at the lateral surface of the distal tip 440. The distal tip 440 further comprises a first side camera 415. The first side camera 415 may be located at the opposite lateral surface of the second side camera 425 such that the two cameras second side camera 425 and first side camera 415 may be pointing at directions essentially opposing to one another. In some other cases, the angle between the second side camera 425 and first side camera 415 may be any workable angle in the range of 10-180 degrees on the circumference of the distal tip 440.

The distal tip 440 may be provided in a diameter of approximately 2.5 to 15 millimeters. The length of the distal tip 440 may be approximately 6.5 to 20 millimeters. In some cases, the distal tip 440 can be connected to the rigid shaft 420, which can be have a diameter of approximately 2.5 to 15 millimeters. In possible embodiments of the disclosed subject matter, the diameter of the rigid shaft 420 may be narrower than the diameter of the distal tip 440. Thus, the distal tip 440 may comprise an inclined surface 435 allowing to connect the distal tip 440 to the rigid shaft 420 provided in a narrower diameter.

In possible embodiments of the disclosed subject matter, the distance between the center of the second side camera 425 and the edge point 430 may be at the same distance between the center of the first side camera 415 and the edge point 465. In possible embodiments of the disclosed subject matter, the distance between the center of the second side camera 425 and the second edge point 430 may be shorter than the distance between the center of the first side camera 415 and the edge point 465. In possible embodiments of the disclosed subject matter, the distance between the center of the second side camera 425 and the first edge point 430 may be longer than the distance between the center of the first side camera 415 and the edge point 465.

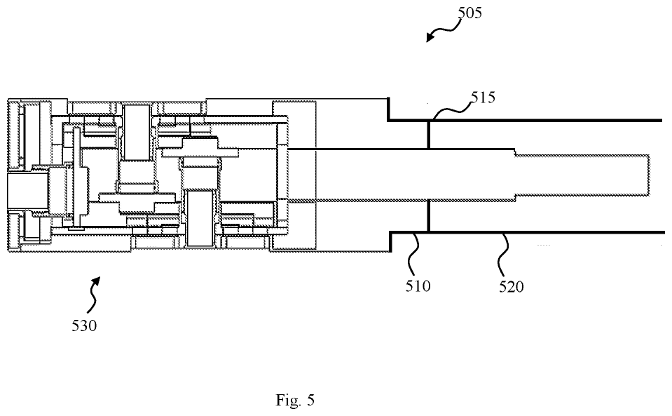

FIG. 5 shows a schematic view of a medical imaging device with a distal tip comprising a graded edge, according to exemplary embodiments of the disclosed subject matter. FIG. 5 shows a medical imaging device 505 comprising a distal tip 530 designed to house cameras and a rigid shaft 520 connected to the distal tip 530. The distal tip 530 comprises a graded edge 510 which allows to connect the distal tip 530 to the rigid shaft 520 at the seamline 515. In some cases, the distal tip 530 comprising a graded edge 510 may be replaced with a distal tip comprising an inclined surface utilized for the purposes of connecting the distal tip to a rigid shaft with a narrower diameter. The graded edge 510 provides a step-like shape for the connection between the distal tip 530 and the rigid shaft 520. In some cases, rigid shaft 520 and graded edge 510 may be connected by an adhesive material which seals the connection at the seamline 515. In some other cases, rigid shaft 520 and graded edge 510 may be connected by soldering. In possible embodiments of the disclosed subject matter, rigid shaft 520 and graded edge 510 may be connected by a screwing mechanism which fastens rigid shaft 520 and distal tip 530 together.

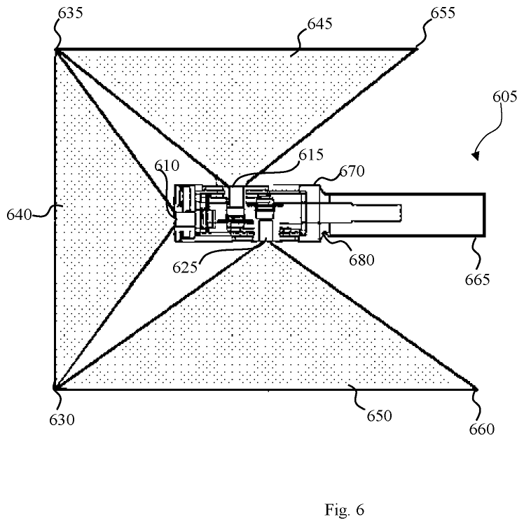

FIG. 6 demonstrates the field of view of three cameras in a medical imaging device with a changing diameter and having a symmetry of overlap between the FOVs, according to exemplary embodiments of the disclosed subject matter. FIG. 6 shows a medical imaging device 605 with a distal tip 670 comprising three cameras, a front camera 610, a second side camera 625 and a first side camera 615. The distal tip 670 also comprises an inclined surface 680 which allows connecting the distal tip 670 to a rigid shaft 665. In some cases, the distal tip 670 may comprise a graded edge which can be connected to the rigid shaft 665.

The distal tip 670 also comprises a front camera 610 provided with a field of view 640, denoted as FOV 640. The FOV 640 can be defined by the imaginary triangle defining the observable view of the front camera 610 and created with the front camera 610, a point 630, and a point 635. Point 635 also outlines one of the edges of the field of view of first side camera 615, denoted as FOV 645. The FOV 645 can be defined by the imaginary triangle defining the observable view of the first side camera 615 and created with the first side camera 615, a point 655 and the point 655. In some cases, FOV 640 may overlap with FOV 645 at the point 635. In some other cases, FOV 654 and FOV 640 may not have any overlap. The point 630 also outlines one of the edges at the field of view of the second side camera 625, denoted as FOV 650. The FOV 650 can be defined at the imaginary triangle defining the observable view of the second side camera 625 and created with the second side camera 625, the point 630 and a point 660. In some cases, FOV 640 may overlap with FOV 650 at the point 630 and the three FOVs, 640, 645 and 650 may not be equal in their symmetry such that an overlap of FOV's can be obtained.

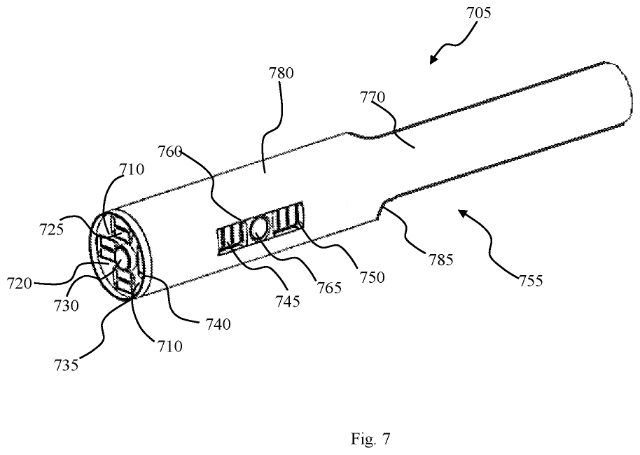

FIG. 7 demonstrates a medical imaging device comprising a one-piece medical imaging device with a changing diameter comprising the optical gear required to the operation of the medical imaging device, according to exemplary embodiments of the disclosed subject matter. FIG. 7 shows a one-piece medical imaging device 705 comprising a rigid shaft 755 without a distal tip. The rigid shaft 755 may function as a multi-camera imaging device designed to house at least one camera. In some cases, the cameras may be at the edge of the rigid shaft 755 located in the front at the planar surface 710. Cameras may also be located at the lateral round surface of the rigid shaft 755. The rigid shaft 755 may comprise a wide section 780 designed to house the optical gear required for the imaging functionality of the medical imaging device. The rigid shaft 755 may also comprise a narrow section 770 designed to convey the wiring required for the optical gear to function. In medical procedures, a person preforming a medical procedure may insert the one-piece medical imaging device 705 into a patient's body such that the wide area 780 may be situated inside the body and the narrow section 770 may be utilized to seal opening at the body, for example in order to prevent from gas to spread out. In some cases, the one-piece medical imaging device 705 may be inserted via a trocar. In such cases, the narrow section 770 may prevent the gas utilized in said medical procedure to spread out via the cannula of the trocar.

The rigid shaft 755 may also comprise an inclined surface 785 designed to bridge between the narrow section 770 and the wide section 780. In some cases, the rigid shaft 755 comprises a narrow section 770 and the wide section 780 may be prepared as one-piece. For example, rigid shaft 755 may be prepared by a molding process. In some cases, the preparation process of the rigid shaft 755 may also comprise a milling process for creating the apertures for the cameras, the rounded surfaces, the planar surfaces, the room for the cameras, and the like.

The rigid shaft 755 also comprises an aperture 760 shaped to house the second side camera 765 and provide the field of view operationally required for the second side camera 765. In some embodiments of the disclosed subject matter, the rigid shaft 755 may comprise a first side camera (not shown) located at the opposite side of the rigid shaft 755. The aperture 760 also houses side illuminator modules 750 and 745 which provides the light source of the side camera 765. In some cases, the light source may be emitted by dedicated endoscope illuminators such as light-emitting diodes, also known as LED. In one embodiment, each illumination module has 1 or more LEDs and may emit different light spectrums.

The rigid shaft 755 may also comprise a front camera 730 situated at the center of a front planar surface 710 which can house the front camera 730 and provide the field of view operationally required for front camera 730. The planar surface 710 also comprise front illuminator modules 720, 725, 735, and 740 which provide the required source of light for front camera 730. In another embodiment, front camera 730 may be situated with a bias to one of the sides of the front planar surface 710.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A medical imaging device, comprising:
a rigid elongated member having a first cross-sectional diameter;
a distal tip directly connected to the rigid elongated member and comprising a front camera located on a front planar surface of the distal tip and a first side camera located on a first later surface of the distal tip; wherein the distal tip has a second cross-sectional diameter, wherein the second cross-sectional diameter is bigger than the first cross-sectional diameter by at least 0.02 millimeter, said distal tip having an inclined surface for maintaining a rigid seamline and gradually bridging a difference between the first cross-sectional diameter and the second cross-sectional diameter.

2. (canceled)

3. (canceled)

4. The medical imaging device of claim 1, wherein the rigid elongated member and the inclined surface of the distal tip are directly connected using a mechanism selected from a group comprising adhesive material, soldering, screwing mechanism and a combination thereof.

5. The medical imaging device of claim 1, wherein the distal tip further comprises a second side camera located on a second lateral surface of the distal tip, wherein the first side camera is closer to the front planar surface than the second side camera.

6. (canceled)

7. The medical imaging device of claim 1, wherein the front camera is located closer to the second lateral surface than to the first lateral surface.

8. The medical imaging device of claim 5, wherein a working distance of each of the front camera, the second side camera and the first side camera is in a range of 1 to 15 millimeters.

9. The medical imaging device of claim 5, wherein the second side camera and the first side camera have a lateral field of view of between 60-160 degrees.

10. The medical imaging device of claim 5, wherein the first side cameras and the second side camera can be pointing perpendicularly to one another, and be positioned essentially 180 degrees apart in a cylindrical surface of the distal tip, in opposite sides of the cylindrical surface of said distal tip.

11. The medical imaging device of claim 5, wherein the first side camera and the second side camera may be positioned in less than 90 degrees apart in the cylindrical surface of the distal tip.

12. The medical imaging device of claim 5, wherein the second side camera and the first side camera are located at the upper half of the distal tip.

13. The medical imaging device of claim 5, wherein the field of view of the second side camera continuous in a sequence manner from a field of view of the front camera.

14. The medical imaging device of claim 5, wherein the field of view of the first side camera continuous in a sequence manner from the field of view of the front camera.

15. The medical imaging device of claim 5, wherein some areas at the field of view of the first side camera and some areas at the field of view of front camera are overlapped.

16. The medical imaging device of claim 1, wherein the front camera is located in the center of the front planar surface.

17. The medical imaging device of claim 5, wherein some areas at the field of view of the second side camera and some areas at the field of view of front camera are overlapped.

18. The medical imaging device of claim 1, wherein the front planar surface creates an angle smaller than 90 degrees from a longitudinal axis of the rigid elongated member.

19. The medical imaging device of claim 1, wherein the distal tip further comprises at least one aperture shaped to house and secure the front camera and the first side camera.

20. The medical imaging device of claim 19, wherein the at least one aperture further comprise opaque walls with transparent apertures located near the front camera and the first side camera.

21. The medical imaging device of claim 1, wherein the distal tip further comprises a front illumination module for illuminating the area captured by the front camera and a lateral illumination module for illuminating the area captured by the first side camera.

22. The medical imaging device of claim 21, wherein the front illumination module comprises two illumination modules located on both sides of the front camera, wherein one illumination module is bigger than the other in case the front camera is not positioned in the center of the front surface.
